SEQUENCE LISTING (6 sequences)

SEQ ID NO: 1 - DNA, 1017 bp, herpes simplex virus 1
atgctcgccgtccgttccctgcagcacctctcaaccgtcgtcttgataacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacccgacgccatgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggtgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcgacagaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccgtggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgttgttccatcatcctctcgggcatcgcagtgcgattgtgttatatcgccgtggtggccggggtggtgctcgtggcgcttcactacgagcaggagatccagaggcgcctgtttgatgtatga

SEQ ID NO: 2 - protein, 338 aa, herpes simplex virus 1
MLAVRSLQHLSTVVLITAYGLVLVWYTVFGASPLHRCIYAVRPTGTNNDTALVWMKMNQTLLFLGAPTHPPNGGWRNHAHICYANLIAGRVVPFQVPPDAMNRRIMNVHEAVNCLETLWYTRVRLVVVGWFLYLAFVALHQRRCMFGVVSPAHKMVAPATYLLNYAGRIVSSVFLQYPYTKITRLLCELSVQRQNLVQLFETDPVTFLYHRPAIGVIVGCELMLRFVAVGLIVGTAFISRGACAITYPLFLTITTWCFVSTIGLTELYCILRRGPAPKNADKAAAPGRSKGLSGVCGRCCSIILSGIAVRLCYIAVVAGVVLVALHYEQEIQRRLFDV

SEQ ID NO: 3 - DNA, 1017 bp, herpes simplex virus 1
atgctcgccgtccgttccctgcagcacctctcaaccgtcgtcttgataacggcgtacggcctcgtgctcgtgtggtacaccgtcttcggtgccagtccgctgcaccgatgtatttacgtggtacgccccaccggcaccaacaacgacaccgccctcgtgtggatgaaaatgaaccagaccctattgtttctgggggccccgacgcacccccccaacgggggctggcgcaaccacgcccacatctcgtacgccaatctgatagcggggagggtcgtgcccttccaggtcccacccgacgccatgaatcgtcggatcatgaacgtccacgaggccgtcaactgtctggagaccctatggtacacacgggtgcgtctggtggtcgtagggtggttcctgtatctggcgttcgtcgccctccaccaacgccgatgtatgtttggcgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtgggctgcgagttgatcgtacgctttgtggccgtgggtctcatcgtcggcaccgctttcatatcccggggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgttgttccatcatcctgtcgggcatcgcaatgcgattgtgttatatcgccgtggtggccggggtggtgctcgtggcgcttcactacgagcaggagatccagaggcgcctgtttgatgtatga

SEQ ID NO: 4 - protein, 338 aa, herpes simplex virus 1
MLAVRSLQHLSTVVLITAYGLVLVWYTVFGASPLHRCIYVVRPTGTNNDTALVWMKMNQTLLFLGAPTHPPNGGWRNHAHISYANLIAGRVVPFQVPPDAMNRRIMNVHEAVNCLETLWYTRVRLVVVGWFLYLAFVALHQRRCMFGVVSPAHKMVAPATYLLNYAGRIVSSVFLQYPYTKITRLLCELSVQRQNLVQLFETDPVTFLYHRPAIGVIVGCELIVRFVAVGLIVGTAFISRGACAITYPLFLTITTWCFVSTIGLTELYCILRRGPAPKNADKAAAPGRSKGLSGVCGRCCSIILSGIAMRLCYIAVVAGVVLVALHYEQEIQRRLFDV

SEQ ID NO: 5 - DNA, 41 bp, Artificial Sequence
  F-primer
tcgaggagaatcctggcccaatgctcgccgtccgttccctg

SEQ ID NO: 6 - DNA, 44 bp, Artificial Sequence
  R-primer
tccgatttaaattcgaattctcatacatcaaacaggcgcctctg

(19) United States
(12) Patent Application Publication
(10) Pub. No.: US 2023/0365981 A1
(43) Pub. Date: Nov. 16, 2023

(54) ANTEROGRADE MONOSYNAPTIC TRANSNEURONAL TRACER SYSTEM WITH GK PROTEIN FUNCTION MANIPULATION

(71) Applicant: WUHAN INSTITUTE OF VIROLOGY, CHINESE ACADEMY OF SCIENCES, Wuhan, Hubei (CN)

(72) Inventors: Min-Hua LUO, Wuhan (CN); Wen-Bo ZENG, Wuhan (CN); Hong YANG, Wuhan (CN); Feng XIONG, Wuhan (CN); Fei ZHAO, Wuhan (CN)

(73) Assignee: WUHAN INSTITUTE OF VIROLOGY, CHINESE ACADEMY OF SCIENCES, Wuhan (CN)

(21) Appl. No.: 17/755,790

(22) PCT Filed: Jun. 22, 2021
(86) PCT No.: PCT/CN2021/101401

§ 371 Date: Jun. 26, 2022

(51) Int. Cl. C12N 15/65; C12N 15/86; C07K 14/005

(52) U.S. Cl. CPC C12N 15/65; C12N 15/86; C07K 14/005; C12N 2800/40; C12N 2710/16622; C12N 2710/16643; C12N 2750/14122; C12N 2750/14143

(57) ABSTRACT

An anterograde monosynaptic transneuronal viral tracer system for mapping the direct postsynaptic targets of neurons in a given brain nucleus comprises a tracer H129-derived recombinant HSV-1 virion; and a helper AAV2/9-derived recombinant AAV2/9 virion; wherein the tracer H129-derived recombinant HSV-1 virion comprises a recombinant HSV-1-H129 viral genome with an impaired gK gene, and a mutant gK protein that pseudotypes the tracer H129-derived recombinant HSV-1 virion; and wherein the helper AAV2/9-derived recombinant AAV2/9 virion comprises a recombinant AAV2/9 viral genome that contains an HSV-1 H129 wild-type gK encoding sequence.

### FIELD OF THE INVENTION

The present invention generally relates to neural biology, and more particularly to an anterograde monosynaptic transneuronal viral tracer system with gK protein function manipulation.

### BACKGROUND OF THE INVENTION

Mapping brain connectome is essential for understanding how the brain works. As the basic unit of neural function, neural circuit serves as the bridge between macroscale structure/function and microscale molecules/signal pathways. However, the structure for many specific functional neural circuits, including the components, connections and distributions, remains to be elucidated. New tracing technology and tracers, especially viral tracers, have contributed to discovery of novel circuits and revealing new features of known canonical circuits.

PCT/CN2016/104882 discloses an anterograde monosynaptic transneuronal tracer system based on human herpes simplex virus type 1 (HSV-1) strain H129 (H129), where H129 virus had a loss of TK function (H129-dTK). However, TK deficiency impairs H129 viral genome replication in neurons, leading to low labeling intensity and tracing efficiency. In addition, H129-dTK-based tracers have the potential of retrograde labeling via axon terminal invasion.

Therefore, there is an imperative need for an anterograde monosynaptic transneuronal tracer system with increased labeling intensity, improved tracing efficiency and reduced incidental retrograde transneuronal transports during tracing.

### SUMMARY OF THE INVENTION

The present invention provides an anterograde monosynaptic transneuronal viral tracer system for mapping direct postsynaptic targets of neurons in a given brain nucleus. In certain embodiments, the anterograde monosynaptic transneuronal viral tracer system for mapping the direct postsynaptic targets of neurons in a given brain nucleus comprises a tracer H129-derived recombinant HSV-1 virion; and a helper AAV2/9-derived recombinant AAV2/9 virion with a recombinant AAV2/9 viral genome containing an HSV-1 wild-type gK encoding sequence; wherein the tracer H129-derived recombinant HSV-1 virion comprises a recombinant HSV-1-H129 viral genome with an impaired gK gene, and a mutant gK protein that pseudotypes the tracer H129-derived recombinant HSV-1 virion.

In certain embodiments of the anterograde monosynaptic transneuronal viral tracer system, in the recombinant HSV-1-H129 viral genome with an impaired gK gene, the impaired gK gene is that gK-coding gene (UL53) is deleted.

In certain embodiments of the anterograde monosynaptic transneuronal viral tracer system, in the recombinant HSV-1-H129 viral genome with an impaired gK gene, the impaired gK gene is that gK-coding gene is replaced with resistance peptide-encoding sequence.

In certain embodiments of the anterograde monosynaptic transneuronal viral tracer system, the resistance peptide-encoding sequence includes Zeo^{R} and Amp^{R}.

In certain embodiments of the anterograde monosynaptic transneuronal viral tracer system, the tracer H129-derived recombinant HSV-1 virion is prepared by:

propagating the recombinant HSV-1-H129 viral genome with an impaired gK gene in Vero cells expressing wild-type H129 gK (gK_{wt}) protein to prepare a seed H129-derived recombinant HSV-1 virion with the gK_{wt} protein (H129-dgK(gK_{wt})); and

propagating the seed H129-dgK(gK_{wt}) in Vero cells expressing the mutant gK (gKₘᵤₜ) protein to prepare the tracer H129-derived recombinant HSV-1 virion (H129-dgK(gKₘᵤₜ)).

In certain embodiments of the anterograde monosynaptic transneuronal viral tracer system, the mutant gK protein has an amino acid sequence represented by SEQ ID NO. 2 in which at least 5 point mutations including A40V, C82S, M223I, L224V, V309M are present.

In certain embodiments of the anterograde monosynaptic transneuronal viral tracer system, the gKₘᵤₜ protein has an amino acid sequence represented by SEQ ID NO. 4.

In certain embodiments of the anterograde monosynaptic transneuronal viral tracer system, the recombinant HSV-1-H129 viral genome with an impaired gK gene further comprises a first expression cassette that contains a first neuronal cell-specific promoter, a first fluorescent protein-encoding sequence.

In certain embodiments of the anterograde monosynaptic transneuronal viral tracer system, the first neuronal cell-specific promoter includes CMV promoter, SV40 promoter, CAG promoter, EF1a promoter, TH promoter, and Syn1 promoter.

In certain embodiments of the anterograde monosynaptic transneuronal viral tracer system, the first expression cassette further comprises a first linker and a second fluorescent protein-encoding sequence, where the first linker is disposed between the first and second fluorescent protein-encoding sequences.

In certain embodiments of the anterograde monosynaptic transneuronal viral tracer system, the first fluorescent protein-encoding sequence encodes a membrane-bound fluorescent protein.

In certain embodiments of the anterograde monosynaptic transneuronal viral tracer system, the recombinant HSV-1-H129 viral genome with an impaired gK gene further comprises a second expression cassette that contains a second neuronal cell-specific promoter, a third fluorescent protein-encoding sequence.

In certain embodiments of the anterograde monosynaptic transneuronal viral tracer system, the second expression cassette further comprises a second linker and a fourth fluorescent protein-encoding sequence, where the second linker is disposed between the third and fourth fluorescent protein-encoding sequences.

In certain embodiments of the anterograde monosynaptic transneuronal viral tracer system, the third fluorescent protein-encoding sequence encodes a membrane-bound fluorescent protein.

In certain embodiments of the anterograde monosynaptic transneuronal viral tracer system, the second neuronal cell-specific promoter includes CMV promoter, SV40 promoter, CAG promoter, EF1a promoter, TH promoter, and Syn1 promoter.

In certain embodiments of the anterograde monosynaptic transneuronal viral tracer system, the viral genome of helper AAV2/9-derived recombinant AAV2/9 virion comprises a third expression cassette that contains a third neuronal cell-specific promoter, the wild-type gK-encoding sequence, a linker peptide-encoding sequence, and a fifth fluorescent protein-encoding sequence.

The present invention provides a method of preparing a tracer H129-derived recombinant HSV-1 virion.

In certain embodiments, the method comprises:

propagating a recombinant HSV-1-H129 viral genome with an impaired gK gene in Vero cells expressing wild-type H129 gK (gK_{wt}) protein to prepare a seed H129-derived recombinant HSV-1 virion with the gK_{wt} protein (H129-dgK(gK_{wt})); and

propagating the seed H129-dgK(gK_{wt}) in Vero cells expressing the mutant gK (gKₘᵤₜ) protein to prepare the tracer H129-derived recombinant HSV-1 virion (H129-dgK(gKₘᵤₜ)).

In certain embodiments of the method, the mutant gK protein has an amino acid sequence represented by SEQ ID NO. 2 in which at least 5 point mutations including A40V, C82S, M223I, L224V, V309M are present.

In certain embodiments of the method, the gKₘᵤₜ protein has an amino acid sequence represented by SEQ ID NO. 4.

The objectives and advantages of the invention will become apparent from the following detailed description of preferred embodiments thereof in connection with the accompanying drawings.

### BRIEF DESCRIPTION OF THE DRAWINGS

Preferred embodiments according to the present invention will now be described with reference to the Figures, in which like reference numerals denote like elements.

FIG. 1 shows schematic illustrations:

(A) genome structure of H129-G4;

(B) genome structure of H129-dgK-G4;

(C) reconstitution and propagation of H129-dgK-G4, i.e. H129-dgK-G4(gK_{wt});

(D) propagation and pseudotype of H129-dgK-G4(gKₘᵤₜ);

(E) genome structure of helper AAVs;

(F) genome structure of helper AAVs for Cre-dependent applications;

(G) mechanism of H129-dgK-G4 monosynaptic transneuronal tracing without helper AAVs; and

(H) mechanism of H129-dgK-G4 monosynaptic transneuronal tracing with helper AAVs.

FIG. 2 shows in vitro and in vivo anterograde monosynaptic transneuronal labeling by H129-dgK-G4 and its helper AAV:

(A) in vitro transneuronal labeling of (a1) H129-G4, (a2) H129-dgK-G4, and (a3) helper AAV+H129-dgK-G4;

(B) protocol of in vivo anterograde monosynaptic transneuronal labeling of H129-dgK-G4 with the helper AAV2/9-mCh-gK;

(C) representative images of the injection site (C), where the white arrows indicate the cells colabeled by mCherry and GFP, representing the potential tracing starter neurons;

(D) representative images of V1 (D); and

(E) representative images of retinas (E) from the same mouse.

FIG. 3 shows the comparison between H129-dgK-G4 and H129-dTK-G4:

(A) schematic genome structure of H129-dTK-G4;

(B) exemplary labeling images of H129-dgK-G4 and H129-dTK-G4 groups;

(C) comparison of labeling intensities between H129-dgK-G4 and H129-dTK-G4 groups as shown in (B), ***, p<0.001;

(D) exemplary labeling images of AAV2/9-mCh-gK+H129-dgK-G4 and AAV2/9-TK-mCh+H129-dTK-G4;

(E) comparison of numbers of GFP⁺Pir neurons in each brain slice between H129-dgK-G4 and H129-dTK-G4 groups as shown in (d), ***, p<0.001.

FIG. 4 shows the comparison of retrograde effects between H129-dgK-G4 (gK_{wt}) and H129-dgK-G4 (gKₘᵤₜ):

(A) schematic diagrams and exemplary images of in vitro comparison of retrograde labeling by axon terminal invasion between H129-dgK-G4 (gK_{wt}) and H129-dgK-G4 (gKₘᵤₜ);

(B) comparison of the numbers of GFP⁺neurons in the soma chamber between H129-dgK-G4 (gK_{wt}) and H129-dgK-G4 (gKₘᵤₜ), ***, p<0.001;

(C) schematic diagrams and exemplary images of in vivo comparison of retrogradely labeling between H129-dgK-G4 (gK_{wt}) and H129-dgK-G4 (gKₘᵤₜ);

(D) comparison of the numbers of GFP⁺neurons in Ect between H129-dgK-G4 (gK_{wt}) and H129-dgK-G4 (gKₘᵤₜ), **, p<0.01.

FIG. 5 shows anterograde monosynaptic tracing of H129-dgK-G4:

(A) schema of the simplified olfactory bulb (OB) projection pathways; OB, olfactory bulb; Pir, piriform cortex; MeA, medial amygdaloid nucleus, anterior part; PMCo, posteromedial cortical amygdaloid nucleus; LEnt, lateral entorhinal cortex;

(B) tracing timeline;

(C) exemplary images of the injection site of OB;

(D)-(G) exemplary images of the downstream projection target brain regions;

(H)-(K) exemplary images of control of H129-dgK-G4 alone.

FIG. 6 shows Cre-dependent anterograde monosynaptic tracing of H129-dgK-G4:

(A) schema of the simplified lateral septal nucleus (LS) projection pathways; LS, lateral septal nucleus; CA1, field CA1 of hippocampus; CA3, field CA3 of hippocampus; MeA, medial amygdaloid nucleus, anterior part;

(B) tracing timeline;

(C) exemplary images of the injection site of LS;

(D)-(F) exemplary images of the downstream projection target brain regions; the boxed areas are presented in the right panels with higher magnification, and the starter neurons labeled by both GFP and mCherry are indicated with white arrows;

(G)-(I) exemplary images of control of 129-dgK-G4 alone.

FIG. 7 shows the quantitative comparison of mPFC-CoA connections between Alzheimer's disease and control mice by anterograde monosynaptic tracing with H129-dgK-G4:

(A) tracing timeline; CoA, cortical amygdaloid nucleus;

(B) exemplary images of the labeled neurons in CoA (indicated with the dashed boxes) of the wildtype C57BL/6 and 3×Tg-AD mice; ACo, anterior cortical amygdaloid nucleus; PMCo, posteromedial cortical amygdaloid nucleus; PLCo, posterolateral cortical amygdaloid nucleus;

(C) quantitative comparison of the number of GFP-labeled (GFP±) neurons in CoA, ***, p<0.001;

(D) quantitative comparison of the ratio of GFP neurons in CoA to mPFC starter neurons, **, p<0.01.

### DETAILED DESCRIPTION OF THE INVENTION

The present invention may be understood more readily by reference to the following detailed description of certain embodiments of the invention.

Throughout this application, where publications are referenced, the disclosures of these publications are hereby incorporated by reference, in their entireties, into this application in order to more fully describe the state of art to which this invention pertains.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, for example, Molecular Cloning: A Laboratory Manual, second edition (Sambrook et al., 1989); Current Protocols in Molecular Biology (F. M. Ausubel et al., eds., 1987).

Adeno-associated virus (AAV) infects humans and some other primate species. The virus is a small (20 nm), replication-defective, and nonenveloped virus. The AAV genome is comprised of single-stranded deoxyribonucleic acid (ssDNA), which is about 4.7 kilobase long. The genome comprises inverted terminal repeats (ITRs) at both ends of the DNA strand, and two open reading frames (ORFs): rep and cap. The former is composed of four overlapping genes encoding Rep proteins required for the AAV life cycle, and the latter contains overlapping nucleotide sequences of capsid proteins: VP1, VP2 and VP3, which interact together to form a capsid of an icosahedral symmetry.

Herpes Simplex Virus type 1 (HSV-1) is a ubiquitous and opportunistic pathogen. The natural neuron tropism and transneuronal transmitting capacity make this virus a potential neural circuit tracer. HSV-1 strain H129 prefers anterograde transneuronal transport, but it also results in not insignificant retrograde transneuronal transport; this complicates the interpretation of tracing results.

PCT/CN2016/104880, titled “ANTEROGRADE MULTI-SYNAPTIC TRANSNEURONAL TRACER”, filed 7 Nov. 2016, is incorporated in its entirety herein.

PCT/CN2016/104882, titled “ANTEROGRADE MONOSYNAPTIC TRANSNEURONAL TRACER SYSTEM” and filed 7 Nov. 2016, is incorporated in its entirety herein.

The inventors of the present invention discovered that TK-deficiency impairs the H129 viral genome replication, resulting in low labeling intensity and tracing efficiency in the application of the anterograde monosynaptic transneuronal tracer system disclosed in PCT/CN2016/104882. Another limitation is that the H129 viral particles with its wild-type surface proteins showed not insignificant retrograde labeling, creating problems in interpretating the mapping results.

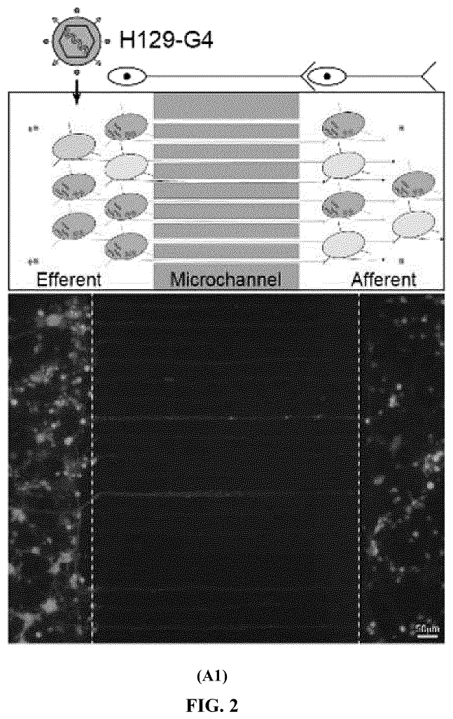

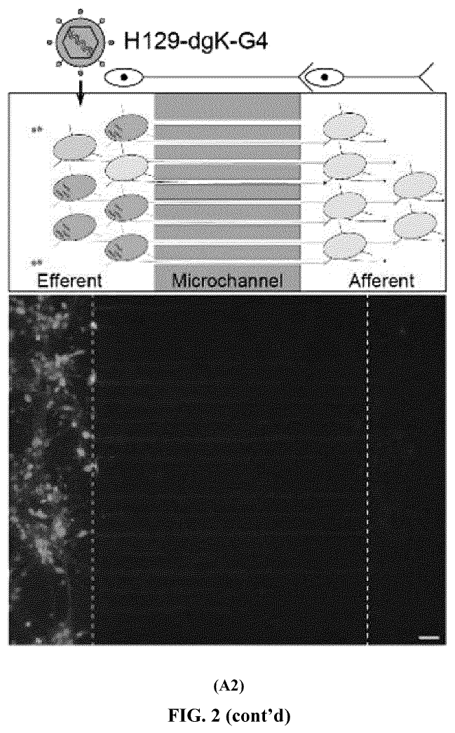

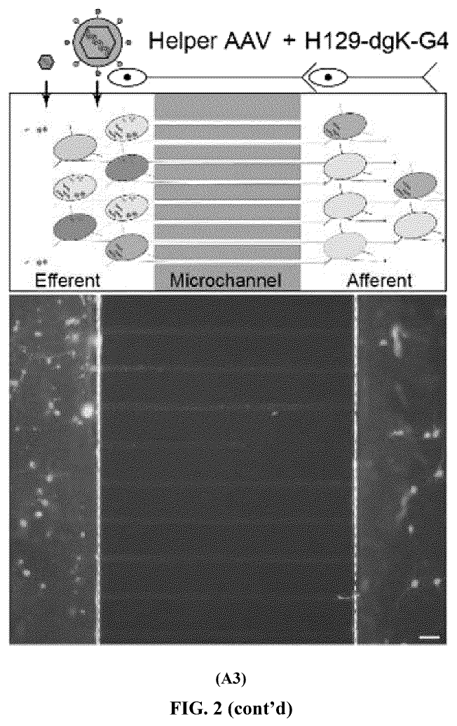

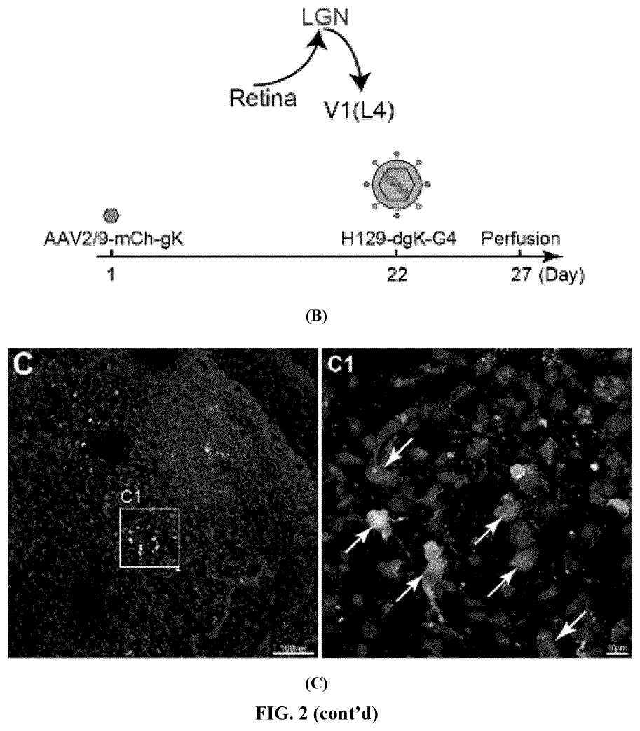

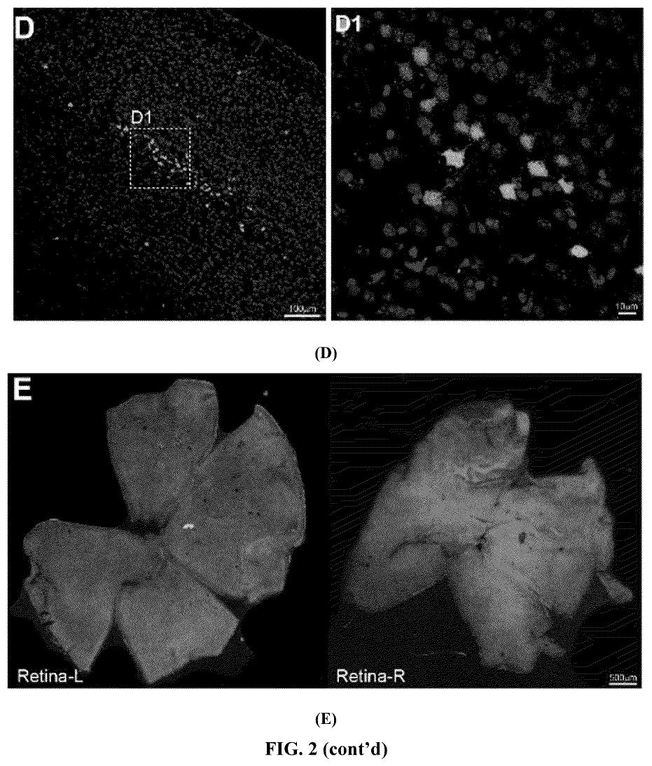

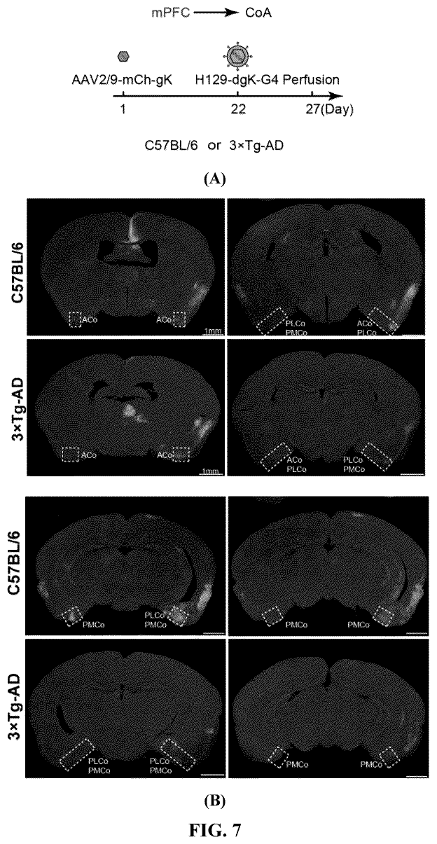

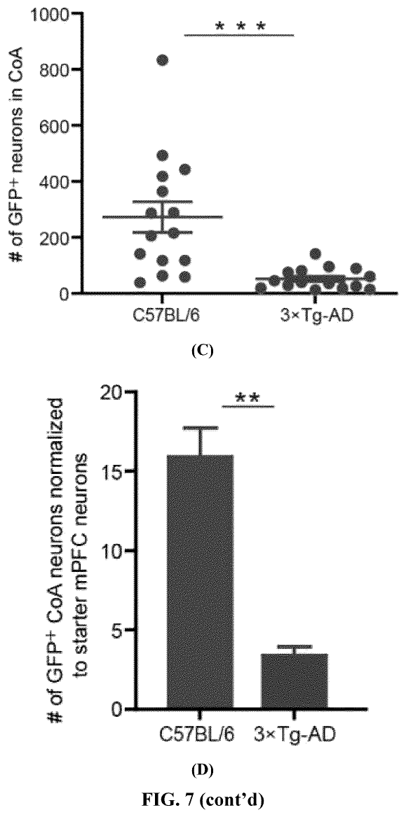

HSV virus entry into all cells involves the coordinated functions of the glycoproteins gD, gB, gH, gL, and gC, and HSV-1 gK is a structural component of the virion particle and functions in virus entry into epithelial cells, cytoplasmic virion envelopment, virion egress and virus-induced cell fusion (David et al., 2012). Through extensive researches using a deficiency/compensation approach, the inventors of the present invention discovered that gK deficiency does not significantly impair H129 viral genome replication, and more importantly, pseudotyping H129 virions with a mutant gK protein minimizes the retrograde lableling in addition to the increase of labeling intensity and tracing efficiency. These results are unexpected in view of the teachings in the previous literatures (e.g. David et al., 2012). For instance, even in VK302 (Vero cell expressing KOS gK protein), gK deletion decreases virus infectivity significantly (FIG. 2 of David et al., 2012), implying that when gK-deleted HSV and AAV helper with mutated gK are co-infected, the production of infectious particles as tracers would be severely impaired. Furthermore, for HSV-1 virions with KOS gK (i.e. mutated gK), both of its retrograde and anterograde activities were impaired (FIG. 7 of David et al., 2012), discouraging anyone from using the mutated gK in a transneuronal tracer.

The present invention provides an anterograde monosynaptic transneuronal viral tracer system for mapping the direct postsynaptic targets of neurons in a given brain nucleus with high labeling intensity and tracing efficiency, and minimized retrograde labeling. Briefly, the anterograde monosynaptic transneuronal viral tracer system comprises a tracer H129-derived recombinant HSV-1 virion and a helper AAV2/9-derived recombinant AAV2/9 virion.

In certain embodiments, the tracer H129-derived recombinant HSV-1 virion contains a recombinant HSV-1-H129 viral genome with an impaired gK gene, and a mutant gK protein that pseudotypes the tracer. The “impaired gK gene” means that when the tracer H129-derived recombinant HSV-1 virion infects neuronal cells by itself alone, the presence of the “impaired gK gene” prevents the resultant virions from egress and transmitting along the axons. In certain embodiments, the “impaired gK gene” includes the modifications to the gK gene and its regulatory elements so that no normal functional gK protein is produced in host cells or incorporated into the resultant virions, where the modifications include deletion, insertion and point-mutations. In certain embodiments, the “impaired gK gene” includes partial or whole replacement of the gK gene by another encoding sequences such as antibiotic-resistant genes used for selection during plasmid or BAC manipulations.

In certain embodiments, the mutant gK protein is a gK protein that has lost or minimized its retrograde activity. In certain embodiments, for a gK protein with an amino acid sequence represented by SEQ ID NO. 2, the mutant gK protein contains in SEQ ID NO. 2 at least 5 point mutations including A40V, C82S, M223I, L224V, V309M. In certain embodiments, the mutant gK protein is represented by SEQ ID NO. 4.

In certain embodiments, in the recombinant HSV-1-H129 viral genome with an impaired gK gene, gK-coding gene (UL53) is deleted (H129-dgK).

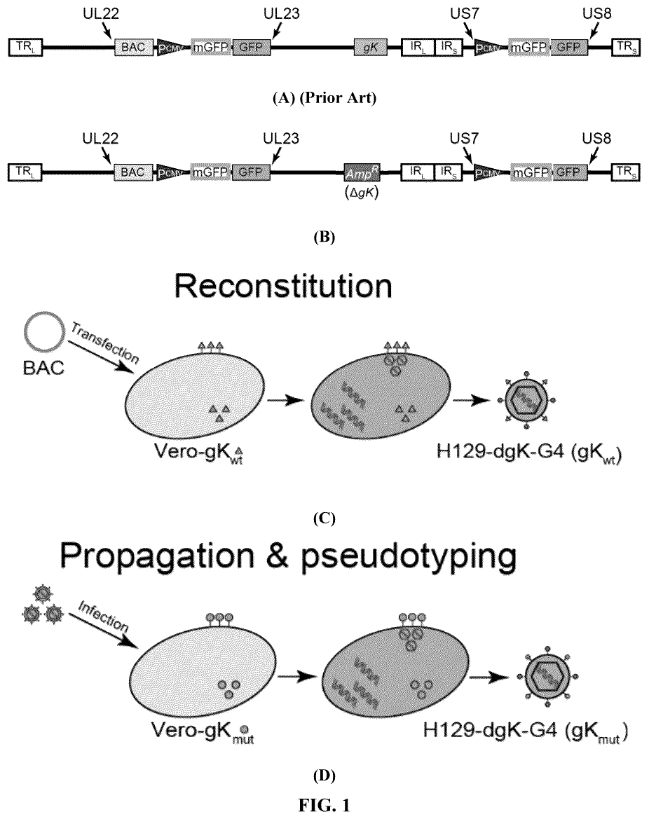

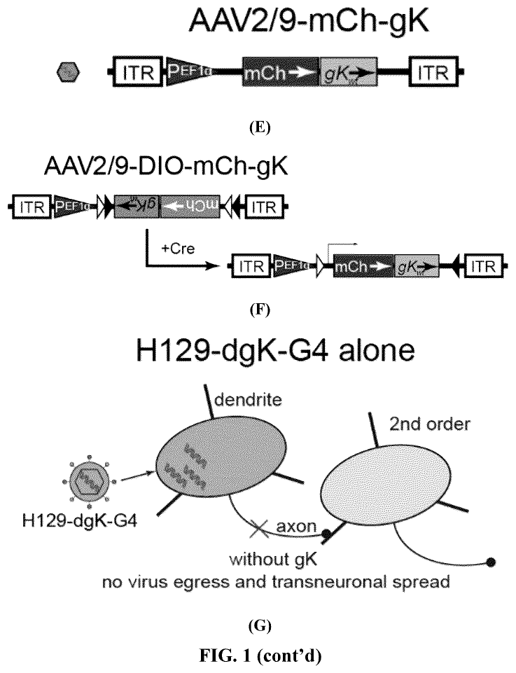

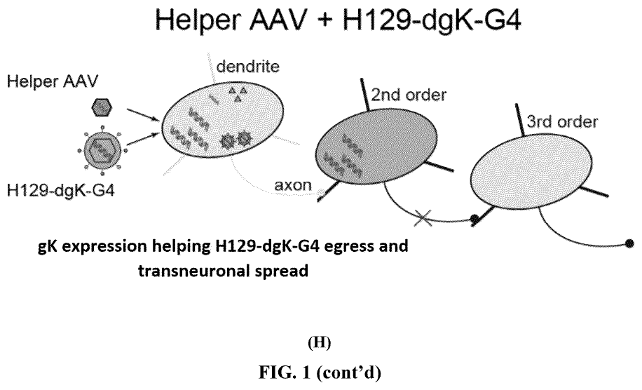

In certain embodiments, in the recombinant HSV-1-H129 viral genome with an impaired gK gene, gK-coding gene is replaced with resistance peptide-encoding sequence. In certain embodiments, the resistance peptide-encoding sequence includes Zeo^{R} and Amp^{R} (FIG. 1B).

In certain embodiments, the tracer H129-derived recombinant HSV-1 virion is prepared using a sequential process. First, using the recombinant HSV-1-H129 viral genome with an impaired gK gene, a seed H129-derived recombinant HSV-1 virion with the wild type gK protein (H129-dgK(gKwt)) is prepared and propagated in the Vero cells expressing wildtype H129 gK (gK_{wt}) protein (e.g., SEQ ID NO. 2); second, the seed virion H129-dgK(gKwt) is propagated in the Vero cells expressing mutant H129 gK (gKₘᵤₜ) protein (e.g., SEQ ID NO. 4) to prepare the tracer virion H129-dgK(gKₘᵤₜ).

In certain embodiments, the recombinant HSV-1-H129 viral genome with an impaired gK gene further comprises a first expression cassette that contains a first neuronal cell-specific promoter, a first fluorescent protein-encoding sequence. In certain embodiments, the first expression cassette further comprises a first linker and a second fluorescent protein-encoding sequence, where the first linker is disposed between the first and second fluorescent protein-encoding sequences. In certain embodiments, the first expression cassette is disposed between UL22 and UL23. In certain embodiments, the first fluorescent protein-encoding sequence encodes a membrane-bound fluorescent protein.

In certain embodiments, the recombinant HSV-1-H129 viral genome with an impaired gK gene further comprises a second expression cassette that contains a second neuronal cell-specific promoter, a third fluorescent protein-encoding sequence. In certain embodiments, the second expression cassette further comprises a second linker and a fourth fluorescent protein-encoding sequence, where the second linker is disposed between the third and fourth fluorescent protein-encoding sequences. In certain embodiments, the second expression cassette is disposed between US7 and US8. In certain embodiments, the third fluorescent protein-encoding sequence encodes a membrane-bound fluorescent protein.

In certain embodiments, the neuronal cell-specific promoter can be any promoter operable in neuronal cells. In certain embodiments, the promoter includes CMV promoter, SV40 promoter, CAG promoter, EF1a promoter, TH promoter, and Syn1 promoter. The first and second neuronal cell-specific promoters can be the same or different.

In certain embodiments, the fluorescent protein-encoding sequence suitable for the present invention can be any fluorescence genes available in the field in the present and future. The fluorescence genes can be wild-type or recombinant derivatives as long as they have no less fluorescent intensity. For example, the fluorescent protein-encoding genes include GFP, sfGFP, EYFP, ECFP, EBFP2, tdTomato, mRFP, mCherry, Ypet, mKo, mkate, etc. In certain embodiments, the first, second, third and fourth fluorescent protein-encoding sequences encode the same fluorescent protein. In certain embodiments, the first, second, third and fourth fluorescent protein-encoding sequences encode different fluorescent proteins.

In certain embodiments, the linker-encoding sequence encodes a linker peptide, where the linker peptide contains at least two adjacent amino acids that are highly inefficient in forming a peptide bond between them. In certain embodiments, the at least two adjacent amino acids are glycine and proline. When the single transcript from the expression cassettes is translated, the first and second (or the third and fourth) fluorescent proteins are generated as separated proteins (not as one fused protein) due to the impedence of peptide bond formation by the linker peptide.

In certain embodiments, the helper AAV2/9-derived recombinant AAV2/9 virion comprises a recombinant AAV2/9 viral genome with a third expression cassette that contains a third neuronal cell-specific promoter, a wild-type gK-encoding sequence, a linker-encoding sequence, and a fifth fluorescent protein-encoding sequence, where the expression of gK from the third expression cassette enables the viral genome of the tracer H129-derived recombinant HSV-1 virions to replicate and fully package in the starting neuronal cells, and then anterograde label the post-synaptic neuronal cells (FIG. 1E-1H). In certain embodiments, the wild-type gK-encoding sequence is represented by SEQ ID NO. 1 or a variant thereof.

In certain embodiments. AAV2/9 has a genome sequence (addgene number 20298).

In certain embodiments, the variants of fluorescent proteins, linker peptide, resistance peptide and gK can be used; where the “variant” is defined as a protein that shares at least 90%, preferably 95%, more preferably 98% or even more preferably 99% identity with an amino acid sequence represented by a corresponding SEQ ID NO number as long as the changes in the variant do not interfere its function.

The present invention also provides a method of preparing a tracer H129-derived recombinant HSV-1 virion.

In certain embodiments, the method comprises:

propagating a recombinant HSV-1-H129 viral genome with an impaired gK gene in Vero cells expressing a wild-type H129 gK (gK_{wt}) protein or a variant thereof to prepare a seed H129-derived recombinant HSV-1 virion with the gK_{wt} protein or variant thereof (H129-dgK(gK_{wt})); and

propagating the seed virion H129-dgK(gK_{wt}) in Vero cells expressing the mutant gK (gKₘᵤₜ) protein to prepare the tracer H129-derived recombinant HSV-1 virion (H129-dgK(gKₘᵤₜ)).

The following embodiments are provided for the purpose of illustrating the application of the principles of the present invention; they are by no means intended to be the coverage of the present invention.

### Embodiment 1

### Cells and Cell Culture

Vero-E6 cell (Vero, ATCC#CRL-1586) was obtained from ATCC, maintained in our laboratory, and tested to be Mycoplasma free. The Vero cell lines stably expressing wildtype glycoprotein K (gK_{wt}) (SEQ ID NO. 2) or mutant glycoprotein K (gKₘᵤₜ) (SEQ ID NO. 4) of H129, namely Vero-gK_{wt} and Vero-gKₘᵤₜ, respectively, were generated by lentivirus transduction. Briefly, gK_{wt} coding gene of H129 (Genebank GU734772.1) (SEQ ID NO. 1) was clone from H129-G4 (Zeng et al. 2017) by PCR (gK_{wt} primers: F-TCG AGG AGA ATC CTG GCC CAA TGC TCG CCG TCC GTT CCC TG (SEQ ID NO. 5), R-TCC GAT TTA AAT TCG AAT TCT CAT ACA TCA AAC AGG CGC CTC TG (SEQ ID NO. 6)), and inserted into the lentivirus vector pCDH-puro to generate the gK_{wt} expressing vector pCDH-puro-gK_{wt} . Then, the gK gene was mutated (A40V, C82S, M223I, L224V, V309M) to generated gKₘᵤₜ expressing vector pCDH-puro-gKₘᵤₜ. The lentiviruses were packaged in human embryonic kidney cell line HEK293T (ATCC, #CRL-11268) as described previously (Yang et al. 2018), and used to transduce Vero cells. The transduced cells were selected with 4 μg/ml puromycin (Sigma) for one week, and then maintained under the pressure of 2 μg/ml puromycin. The resulted cell lines were designated as Vero-gK_{wt} and Vero-gKₘᵤₜ, respectively. All these cells were cultured with Dulbecco's modified Eagle medium (DMEM, Gibco/Life Technologies) containing 10% fetal bovine serum (FBS, Gibco/Life Technologies) and penicillin (100 U/ml)-streptomycin (100 μg/ml) (Gibco/Life Technologies).

Fetal mouse cortical neurons were isolated and cultured as described previously (Dong et al. 2020; Yang et al. 2018). Briefly, the cerebral cortex was dissected from the forebrain of C57BL/6 mouse fetuses at embryonic day 18.5 (E18.5), and then dissociated with 0.25% trypsin (Gibco/Life Technologies)/DNase I (Sigma) for 15 min at 37° C. After being washed with Ca²⁺/Mg²⁺free Hank's Balanced Salt Solution (HBSS) (Gibco/Life Technologies), the isolated neurons were resuspended, plated in microfluidic plates, and cultured in Neurobasal medium (Gibco/Life Technologies) supplemented with B27 (2%) (Gibco/Life Technologies), GlutaMAX (25 μM) (Gibco/Life Technologies) and penicillin (100 U/ml)-streptomycin (100 μg/ml). The medium was refreshed every 2 days.

All cells were cultured at 37° C. in a humidified atmosphere containing 5% CO_{2.}

### Embodiment 2

### Statistical Analysis

Each experiment was performed in triplicate, and the results were presented as means ±SEM (Standard Error of the Mean) from at least three independent experiments or animals. Appropriate statistical tests were applied in the data analysis, including Student's t test or linear mixed-effect model (LME) analysis. LME has been widely used to analyze correlated data such as clustered data. Differences were considered to be significant when the p value was <0.05.

### Embodiment 3

### Construction and Propagation of the Recombinant Viruses: H129-dgK-G4, H129-dTK-G4, and Helper AAVs

H129-dgK-G4 was derived from the previously introduced H129-G4 (FIG. 1A) (Zeng et al. 2017). Using the bacterial artificial chromosome (BAC) and homologous recombination technique, the gK gene (UL53) was knocked out by replacing it with an Ampicillin resistant gene (Amp^{R}) (FIG. 1B). The obtained recombinant BAC DNA was transfected into Vero-gK_{wt} to reconstitute the recombinant virus coated with gK_{wt}, labeled as H129-dgK-G4(gK_{wt}) (FIG. 1C). The reconstituted virus was further propagated in Vero-gKₘᵤₜ to generate the gKₘᵤₜ pseudotyped recombinant virus H129-dgK-G4(gKₘᵤₜ) (FIG. 1D). H129-dgK-G4(gKₘᵤₜ) is deficient with gK in the genome and is pseudotyped with gKₘᵤₜ on the envelope. To simplify the labeling, all H129-dgK-G4 in the present application represents the gKₘᵤₜ pseudotyped virus H129-dgK-G4(gKₘᵤₜ), unless specifically indicated. The H129-derived tracer virions were propagated following our previously published protocol (Yang et al. 2020), and the ready-to-use H129-dgK-G4 typically reaches an average titer of 5×10⁸ pfu/ml.

For monosynaptic tracing, the helper AAV viruses of AAV2/9-mCh-gK (FIG. 1E) and AAV2/9-DIO-mCh-gK (FIG. 1F), expressing mCherry and gK_{wt} constitutively or via a Cre-dependent manner, were constructed and packaged, respectively as described previously (Yang et al. 2020; Zeng et al. 2017).

gK deficiency abolishes viral egress, transneuronal transmission, and infection of H129-dgK-G4 (FIG. 1G). In the same neuron, helper AAV compensatorily expresses gK_{wt} protein that resides on the envelope of H129-dgK-G4 virion. Then the gK_{wt}-compensated H129-dgK-G4 can egress and transmit one step down to the postsynaptic neurons that are then labeled by mGFP/GFP expressed in situ (FIG. 1H).

Similarly, H129-dTK-G4 and the corresponding helper AAV (AAV2/9-TK-mCh) were generated and propagated/packaged as described previously (Yang et al. 2020; Zeng et al. 2017).

### Embodiment 4

### Microfluidic Assay

The microfluidic plate has been introduced previously and was fabricated following the described protocol (Zeng et al. 2017). In brief, it contains two isolated chambers connected by multiple microchannels (700 μm long, 10 μm wide, and 3 μm deep), which allows only the axons to grow through but not the somas or dendrites. Quality control of the microfluidic plates was performed using 5 randomly selected plates from each fabrication batch (50 plates) to examine the potential inter-chamber leakage. Vero cells (5×10⁵) were cultured in one chamber (day 1), where cells cannot grow through the microchannels to reach the opposite chamber, and H129-G4 (1×10⁶ pfu) was added to the opposite chamber on day 5. To avoid virus diffusion, less medium volume was maintained in the virus-inoculating chamber to achieve lower hydrostatic pressure. On day 8, the plates were examined for GFP signal caused by H129-G4 infection of Vero cells, which indicates possibly inter-chamber leakage. Then the certified batch of microfluidic plates was applied for further experiments only when no leakage occurred in any tested plates.

For microfluidic assays, fresh isolated fetal mouse cortical neurons (1×10⁶) were plated into both chambers (on day 1 and day 5 respectively) or one chamber (on day 1) of the microfluidic plate. The viruses were added into the indicated chamber with lower hydrostatic pressure. To test the transneuronal transmission, AAV2/9-mCh-gK (1×10¹⁰ vg, when indicated) were added to the efferent chambers on day 14, and H129-derived tracers (1×10⁶ pfu, as indicated) were added to the same chamber on day 20. GFP positive neurons were examined 2 days after adding the H129-derived tracers. To test the retrograde labelling caused by axon terminal invasion, H129-derived tracers (1×10⁶ pfu) were added to the axon terminal chamber on day 14. GFP positive neurons were examined 1 day after adding the H129-derived tracers.

As shown in FIG. 2A1, consistent with previously published results (Zeng et al. 2017), the polysynaptic tracer H129-G4 labeled neurons in both chambers, indicating it replicated in the efferent neurons. then transmitted to the downstream afferent neurons through the axons in microchannels, and label the downstream neurons. H129-dgK-G4 alone well labeled the efferent neurons, but failed to transmit to and label the downstream afferent neurons (FIG. 2A2). When the helper AAV2/9-mCh-gK (1×10¹⁰ vg) was administrated 7 days prior to the H129-dgK-G4 inoculation, AAV2/9-mCh-gK and H129-dgK-G4 coinfection labeled many efferent neurons with mGFP/GFP and mCherry (merged as yellow). And notably, some of the downstream afferent neurons were clearly labeled with GFP, suggesting H129-dgK-G4 anterogradely transmitted to the postsynaptic neurons with the helper AAV compensatorily providing gK (FIG. 2A3), but not alone by H129-dgK-G4 itself (FIG. 2A2).

### Embodiment 5

### 5.1 Intracranial Injection of the Viral Tracers

Wildtype C57BL/6 mice were purchased from Beijing Vital River Laboratory Animal Technology company. GAD2-Cre transgenic mice, which specifically express Cre recombinase under the control of glutamic acid decarboxylase promoter, were provided by the Laboratory Animal Resource Center at the Chinese Institute for Brain Research, Beijing (CIBR), and the 3×Tg-AD mice showing symptoms of Alzheimer's disease were provided by the Department of Physiology, School of Basic Medicine and Tongji Medical College, Huazhong University of Science and Technology. The parameters of the mouse brain regions were determined according to the Mouse Brain Atlas by the mediolateral (ML), anteroposterior (AP) and dorsoventral (DV) distances to Bregma. The indicated viral tracers were intracranially administrated into the target brain region using a motorized stereotaxic injector (Stoelting) under anesthesia.

Helper AAVs (1.0×10¹² vg/ml, 100-150 nl) and H129-derived tracers (5.0×10⁸ pfu/ml, 100-150 nl) were sequentially injected into the same location of the indicated brain regions on day 1 and 22, respectively. On day 27, animals were anesthetized and perfused with sterile normal saline and 4% paraformaldehyde (PFA) solution, and the whole brain was carefully collected. The obtained brains were fixed with 4% PFA, dehydrated in 30% sucrose, and stored at 4° C. for further cryosection and imaging. When indicated, the same amounts of H129-derived tracers were injected into the indicated brain regions alone, and the brains were collected at 5 days post-injection.

### 5.2 Cryosection and Imaging

After fixation and dehydration, the obtained brains were coronally cryo-sectioned to 40 μm-thick slices using a microtome (HM550, Thermo/Life Technologies). The only staining is to show cell nuclei by counterstaining with DAPI (Cat. #10236276001, Roche), and all the GFP signals are natural origins without any signal amplification. All images were obtained using a Nikon's AIR MP+ confocal microscope equipped with a fast high-resolution galvanometer scanner.

To count the labeled neuron number in the indicated brain regions of each mouse, the coronal brain slices at similar positions were observed with an interval of 160 μm (one from continuous 5 40μm-thick slices), and the labeled neurons at the indicated brain regions were counted. To measure the labeling intensity of GFP positive neurons, three slices at similar positions from the indicated brain regions of the same brain were selected, and the GFP intensity was quantified by ImageJ software v1.60 (NIH, USA). By this method, the quantified value of mean GFP intensity of the labeled cells was shown as the arbitrary unit (AU), which was calculated as IntDen/Area (IntDen, Integrated Density of GFP-labeled neurons; area, the total area of GFP-labeled neurons).

### 5.3 Results

Similar to the in vitro result obtained in the microfluidic plates, H129-dgK-G4 also displayed the capability of anterograde monosynaptic tracing in vivo stringently depending on helper AAV compensating gK. When applied alone, H129-dgK-G4 only infected and labeled neurons around the injection site, but did not spread from the tested brain regions, including the primary motor cortex (M1), auditory cortex (Au), and dentate gyrus (DG) of wildtype C57BL/6 mice. However, upon the presence of gK expressing helper AAV, H129-dgK-G4 achieved anterograde monosynaptic tracing in vivo. As shown in FIG. 2B, AAV2/9-mCh-gK (1.0×10¹² vg/ml, 100 nl) and H129-dgK-G4 (5.0×10⁸ pfu/ml, 100 nl) were sequentially injected into the lateral geniculate nucleus (LGN, AP: −2.30 mm; ML: −2.13 mm; DV: −2.75 mm) of wildtype C57BL/6 mice on day 1 and day 22, respectively, and brains were collected on day 27. Neurons expressing both niCherry and GFP (merged as yellow) were observed around the injection site LGN (FIG. 2C, indicated with white arrows), representing the potential starter neurons for initial transmission. In the downstream brain region V1, GFP-labeled neurons were only observed in Layer IV (L4), which is directly innervated by LGN, indicating that H129-dgK-G4 anterogradely transmits through one order to the postsynaptic neurons with the helper AAV compensating gK_{wt} (FIG. 2D), H129 has been shown to have potential retrograde transmission. However, in the retina, which is a direct upstream region of the LGN, no GFP positive neuron was observed, indicating there is no retrograde transmission and labeling of H129-dgK-G4 in the experimental condition (FIG. 2E).

Altogether, these data suggest that H129-dgK-G4 can transmit to and label downstream neurons in an anterograde monosynaptic manner, depending on helper AAV compensating gK_{wt}.

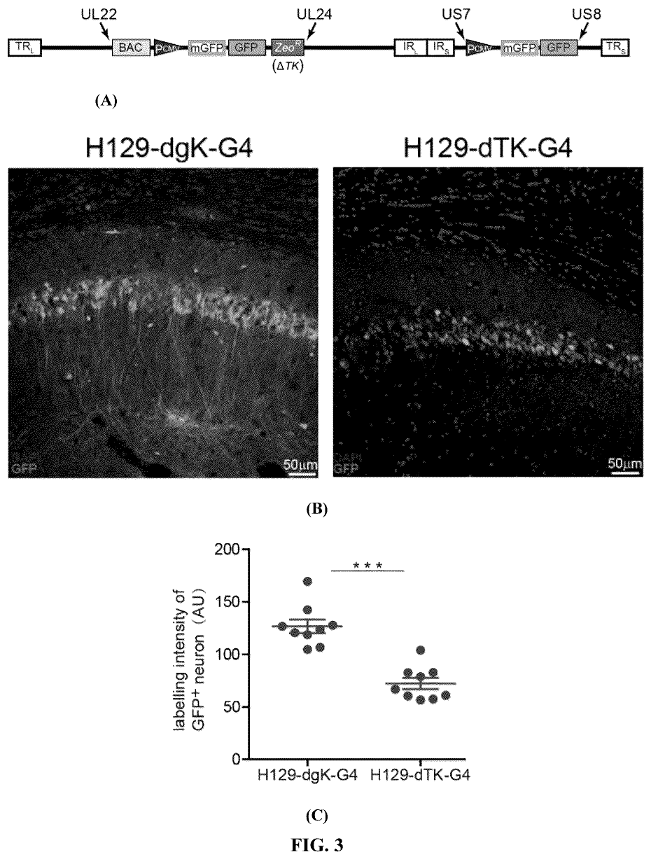

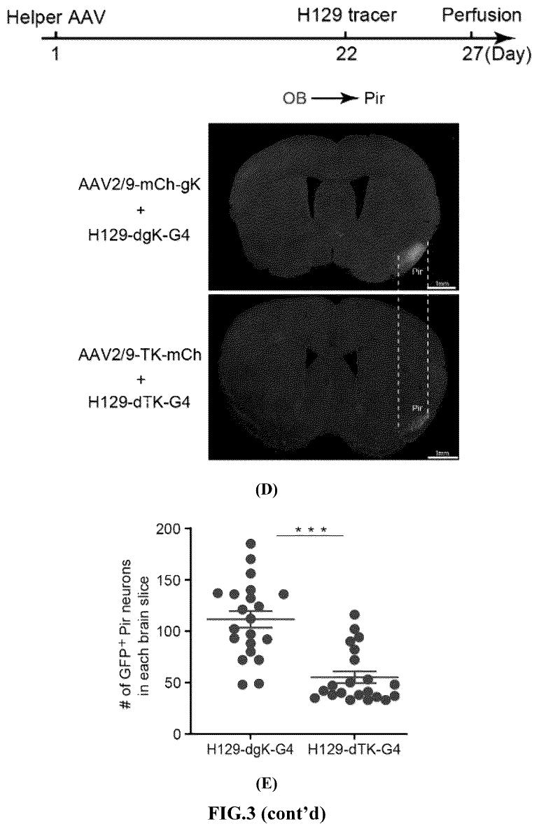

We have previously introduced H129-dTK-tdT, and then an updated version H129-dTK-T2 with an improved labeling intensity by adding an extra tdTomato expression cassette (Zeng et al. 2017). We also generated H129-dTK-G4 (FIG. 3A), whose labeling intensity is similar to H129-dTK-T2. The strategy of these monosynaptic tracers is dependent on thymidine kinase (TK) deficiency. TK deficiency impairs viral genome replication in neurons, and limits viral protein synthesis, as well as the fluorescent proteins reporter, which leads to low labeling intensity in the postsynaptic neurons. This is an intrinsic drawback of all TK deficient tracers.

The structural gene gK was knocked out instead of TK required for viral genome replication. H129-dgK-G4 and H129-dTK-G4 were both derived from H129-G4, only differently deficient with gK or TK, respectively. The labeling intensity and tracing efficiency were quantitated and compared between H129-dgK-G4 and H129-dTK-G4. For H129-dgK-G4 or H129-dTK-G4 along with the helper AAV, only the H129-derived deficient tracers, but not the helper AAVs, transmit to and label the postsynaptic neurons. Thus, to mimic the similar condition, we injected H129-dgK-G4 or H129-dTK-G4 alone into the CA1 (AP: −2.18 mm; ML: −1.00 mm; DV: −1.50 mm) of wildtype C57BL/6 mice with the same dosage (5×10⁸ pfu/ml, 100 nl). As shown in the exemplary images (the original GFP signal and DAPI counterstaining), H129-dgK-G4 labeled neurons around the injection site with a stronger labeling intensity than H129-dTK-G4 (FIG. 3B). Quantitation analysis of the fluorescence brightness of the labeled neurons showed that the average labeling intensity of H129-dgK-G4 is 1.76-fold higher than that of H129-dTK-G4 (127±7 AU vs 72±5 AU) (FIG. 3C). Therefore, H129-dgK-G4 has stronger labeling intensity.

The competent replication and increased labeling intensity of H129-dgK-G4 can potentially improve the tracing efficiency by visualizing more labeled postsynaptic neurons. To assess the tracing efficiency, H129-dgK-G4 and H129-dTK-G4 were applied in mapping the olfactory pathways, and the labeled postsynaptic neurons were counted and analyzed. H129-dgK-G4 or H129-dTK-G4 (5.0×10⁸pfu/ml, 150 nl) was injected into the olfactory bulb (OB, AP: +4.28 mm; ML: −0.50 mm; DV: −2.50 mm) of wildtype C57BL/6 mice along with the corresponding helper AAV, AAV2/9-mCh-gK or AAV2/9-TK-mCh (1.0×10¹² vg/ml, 150 nl), respectively. Both tracers anterogradely transmitted to the downstream brain regions and labeled the neurons, represented by the piriform cortex (Pir) (FIG. 3D). We counted GFP-labeled (GFP⁺) Pir neurons in the position-matching brain slices of each mouse brain (7 slices per mouse, and 3 mice per group). An average of 112±11 GFP⁺neurons was observed in the Pir of each slice by H129-dgK-G4 tracing, while only half amount of GFP⁺Pir neurons (56±13) was observed by H129-dTK-G4 tracing (FIG. 3E). Compared to H129-dTK-G4, H129-dgK-G4 has doubled (2.00-fold) tracing efficiency, which is an extra bonus of the new tracer.

Taken together, H129-dgK-G4 achieves a significant increase in both the labeling intensity and tracing efficiency.

H129 has axon terminal invasion, which makes the neurons in the upstream regions be retrogradely labeled by H129-derived anterograde tracers. Our previous study showed retrograde labeling ratio of the H129-derived tracer is associated with the brain regions, viral titer, administration dosage, and the tracing duration (Zeng et al. 2017). Although carefully optimizing these experimental parameters can limit the potential retrograde labeling, a fundamental solution is still required to overcome the natural viral property of axon terminal invasion, so that to further minimize potential retrograde labeling for a higher tracing specificity.

The Vero cell line stably expressing the mutant gK (gKₘᵤₜ, A40V, C82S, M223I, L224V, V309M) was generated, namely Vero-gKₘᵤₜ, which was used to propagate the gKₘᵤₜ pseudotyped H129-dgK-G4, the final product of the tracer.

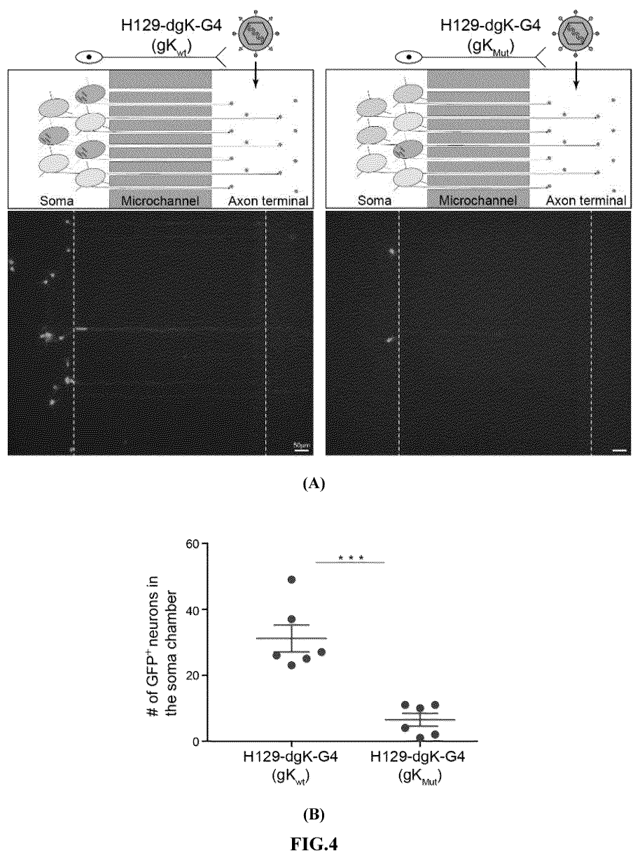

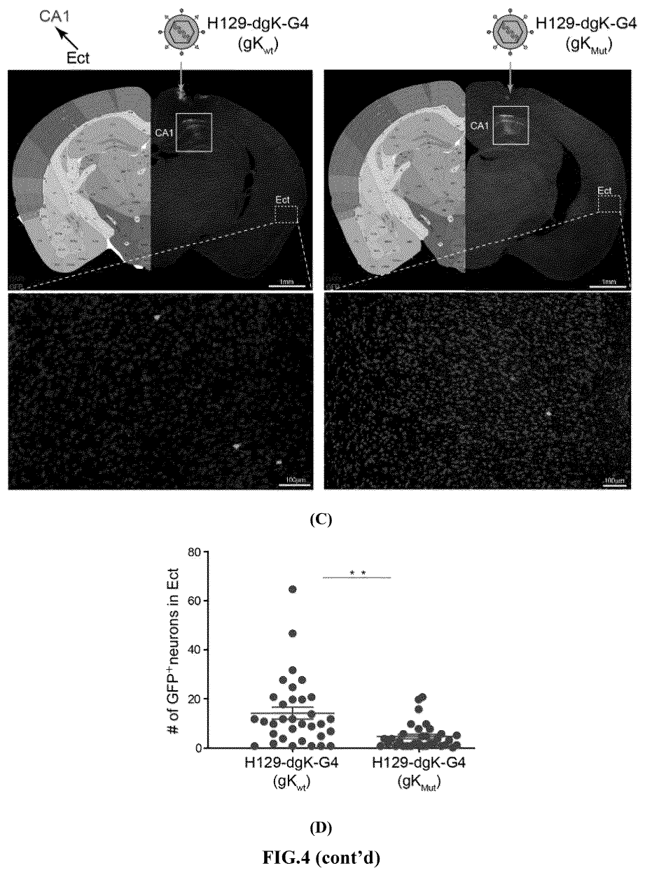

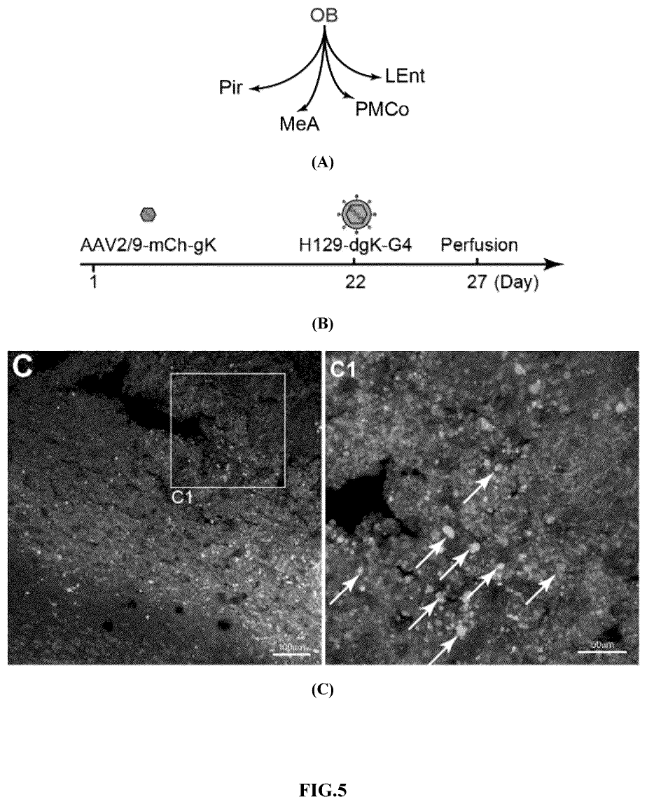

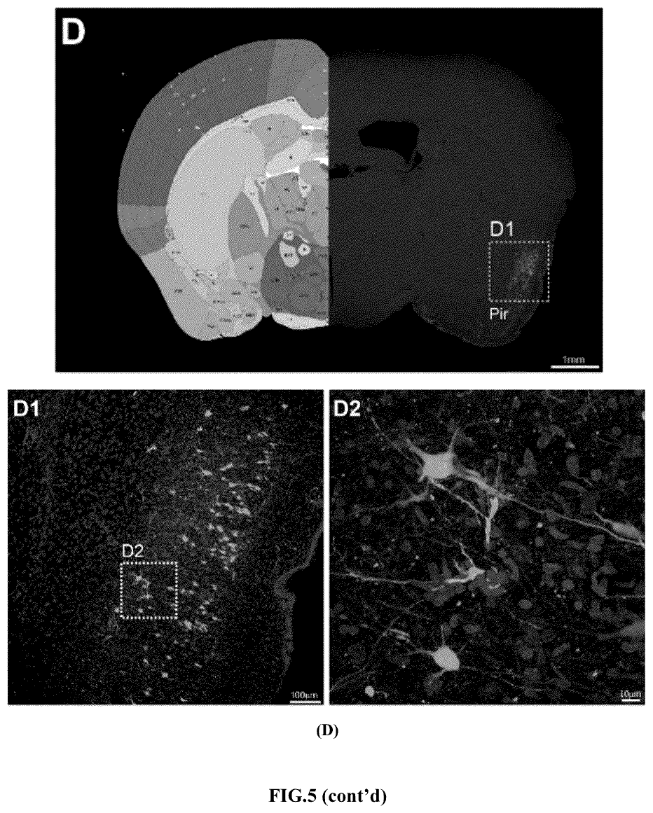

The retrograde labeling ratio was quantitated and compared between H129-dgK-G4 tracers pseudotyped with gKₘᵤₜ (H129-dgK-G4(gKₘᵤₜ)) and coated with gK_{wt} (H129-dgK-G4(gK_{wt})). In vitro, 1×10⁶ neurons were cultured in one chamber (soma chamber) of each microfluidic plate, and the axons grew through the microchannels reaching the other chamber (axon terminal chamber). H129-dgK-G4(gKₘᵤₜ) or H129-dgK-G4(gK_{wt}) (1×10⁶ pfu) was added to the axon terminal chamber. At 1 day post infection (dpi), GFP-labeled neurons, caused by viral tracer infection via axon terminal invasion, were monitored and counted in the soma chambers (FIG. 4A). An average of 31±4 neurons in the soma chambers were retrogradely labeled by H129-dgK-G4(gK_{wt} ), while only 7±2 by H129-dgK-G4(gKₘᵤₜ). Therefore, gKₘᵤₜ pseudotyping dramatically reduced the retrograde labeling of H129-dgK-G4 tracer by 77% in vitro (FIG. 4B).

Similarly, gKₘᵤₜ pseudotyping decreased the retrograde labeling In vivo. H129-dgK-G4(gKₘᵤₜ) or H129-dgK-G4(gK_{wt}) (5.0×10⁸ pfu/ml, 100 nl) was injected into the CA1 (AP: −2.18 mm; ML: −1.00 mm; DV: -1.50 mm) of wildtype C57BL/6 mice. Both tracers nicely labeled the neurons around the injection site (FIG. 4C, upper panel). A few GFP positive neurons were observed in the ectorhinal cortex (Ect), which is an upstream brain region innervating CA1, indicating the retrograde labeling by virus via axon terminal invasion from CA1 (FIG. 4C, lower panel). By carefully counting the GFP-labeled Ect neurons in the position-matching brain slices from each mouse, it is shown that H129-dgK-G4(gK_{wt}) averagely labeled 13±2 Ect neurons in each brain slices, while H129-dgK-G4(gK_{wt}) labeled only 3±3 (FIG. 4D). Thus, gKₘᵤₜ pseudotyping the H129-dgK-G4 achieved a 77% decrease of the retrograde labeling.

Notably, retrograde labeling of H129-dgK-G4(gKₘᵤₜ) was only observed in CA1, but not in any other tested brain regions, including olfactory bulb, primary motor cortex, infralimbic cortex, dentate gyrus, Auditory cortex, primary visual cortex, and median raphe nucleus (data not shown). And low level of retrograde labeling from CA1 occurred only at a high injection dosage (5.0×10pfu/ml, 100 nl) of H129-dgK-G4(gKₘᵤₜ). When gKₘᵤₜ pseudotyped H129-dgK-G4 was injected at any lower dosages (1.0×10⁸ or 2,5×10⁸ pfu/ml, 100 nl) or other tested brain regions, no retrograde labeling was observed (data not shown),

Taken together, both the in vitro and in vivo assessments confirm that gKₘᵤₜ pseudotyping reduces the incidence of the retrograde labeling of H129-dgK-G4. It represents a more anterograde-specific monosynaptic tracer with less retrograde labeling, and limits potential misleading interpret. Thus, all H129-dgK-G4 used throughout the application were propagated in Vero-gKₘᵤₜ cell and is gKₘᵤₜ pseudotyped H129-dgK-G4 tracer.

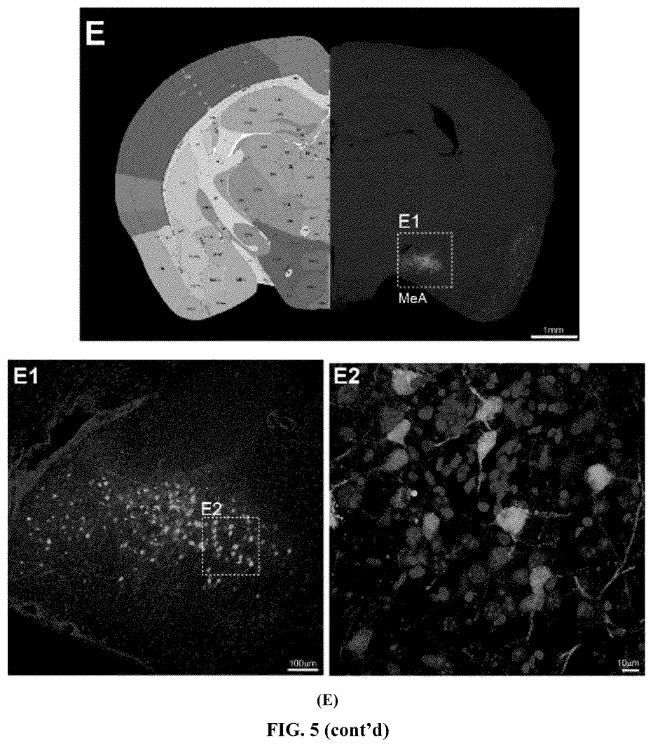

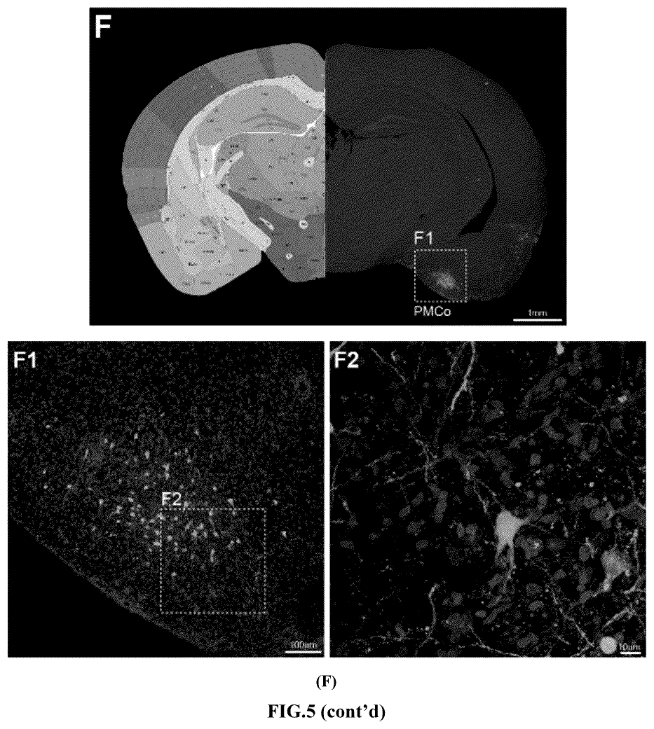

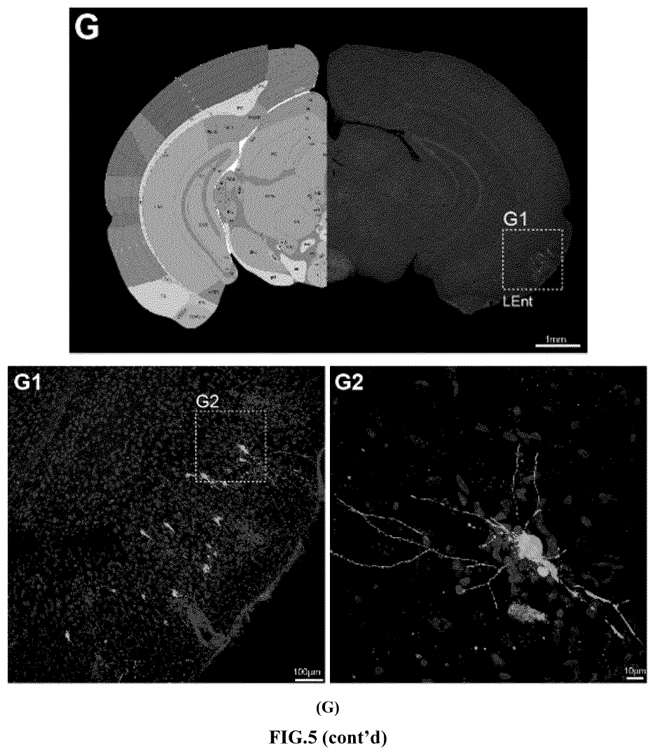

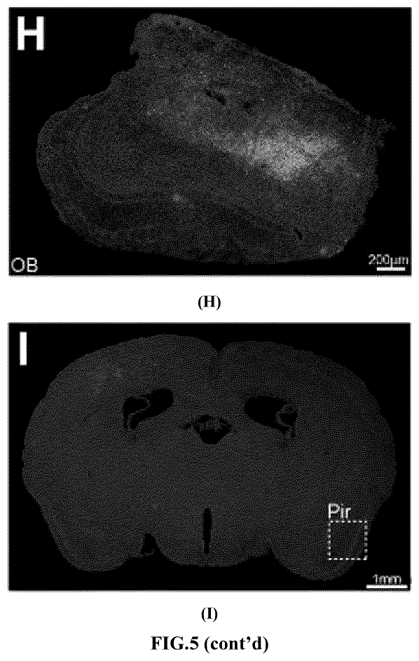

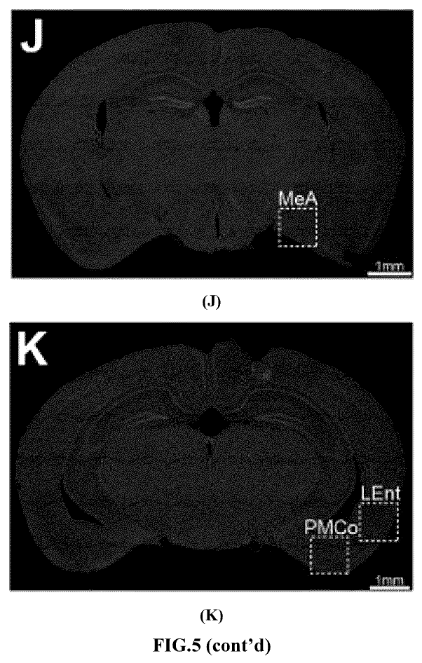

The features of gKₘᵤₜ pseudotyped tracer H1.29-dgK-G4 include stronger labeling intensity, higher tracing efficiency, and higher anterograde specificity as described above. The anterograde monosynaptic tracing of H129-dgK-G4 was firstly tested in the olfactory bulb (OB) circuit (FIG. 5A). AAV2/9-mCh-gK (1,0×10¹² vg/ml, 150 nl) and H129-dgK-G4 (5.0×10⁸ pfu/ml, 150 nl) were sequentially injected into the OB (AP: −4.28 mm; ML: −0.50 mm; DV: −2.50 mm) of wildtype C57BL/6 mice on day 1 and day 22, respectively, and brains were obtained on day 27 (FIG. 5B). The labeling change with time was tested, a desirable labeling intensity and efficiency were observed on day 27, thus the brains were collected on day 27 in all the experiments. Neurons labeled with mCherry and GFP (merged as yellow, indicated with white arrows) directly observed around the injected site, which were coinfected by both viruses (FIG. 5C). They also represented the potential starter neurons for transmission initiation, from which the gK compensated H129-dgK-G4 transmitted to and labeled the postsynaptic neurons in the downstream brain regions. The GFP-labeled neurons were observed in the representative OB projecting regions, such as piriform cortex (Pir) (FIG. 5D), medial amygdaloid nucleus, anterior part (MeA) (FIG. 5E), posteromedial cortical amygdaloid nucleus (PMCo) (FIG. 5F), and lateral entorhinal cortex (LEnt) (FIG. 5G). Notably, All these GFP⁺neurons were labeled with strong intensity and directly visible without signal amplification by immunostaining. Injection of H129-dgK-G4 alone only labeled neurons around the injection sites (OB) (FIG. 5H), but not the connected regions, indicating neither non-specific transmission nor retrograde labeling occurred under this experimental condition (FIG. 51-K), which guaranteed the specificity of the anterograde monosynaptic tracing of H129-dgK-G4.

To precisely map the neuronal circuits, output information from a specific type of neuron is also required. In mice, the Cre/lox recombination system is the most widely used approach to access specific neuron types. So we further tested the starter-specific anterograde monosynaptic tracing ability of the H129-dgK-G4 system in GAD2-Cre transgenic mice, which specifically express Cre recombinase in neurons with glutamic acid decarboxylase 2 (GAD2). AAV2/9-DIO-mCh-gK was applied as the helper virus to assist the Cre-dependant anterograde monosynaptic tracing of H129-dgK-G4 from a specific neuron type. Controlled by the double floxed inverted orientation (DIO) Cre-On system, AAV2/9-DIO-mCh-gK expresses mCherry and gK only in the presence of Cre recombinase, and therefore assists H129-dgK-G4 monosynaptic transmission only from the specifical Cre expressing neurons.

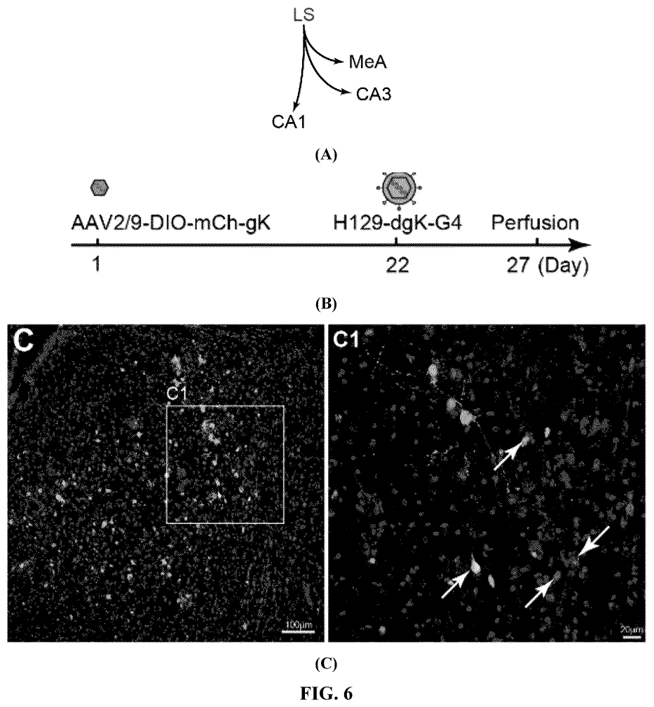

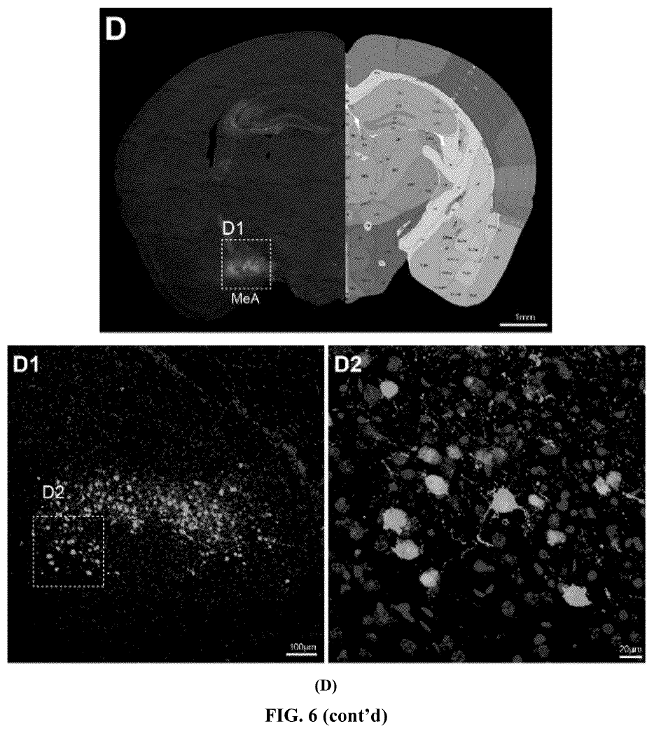

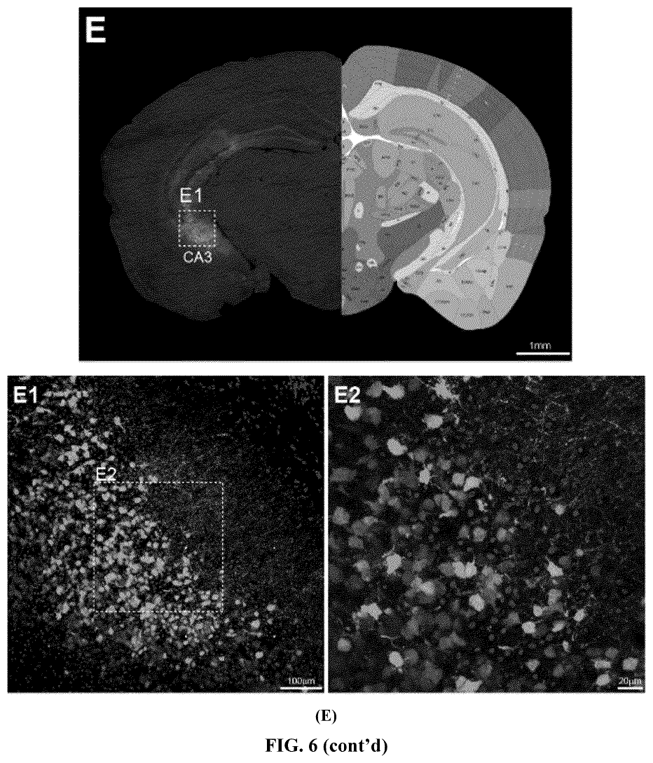

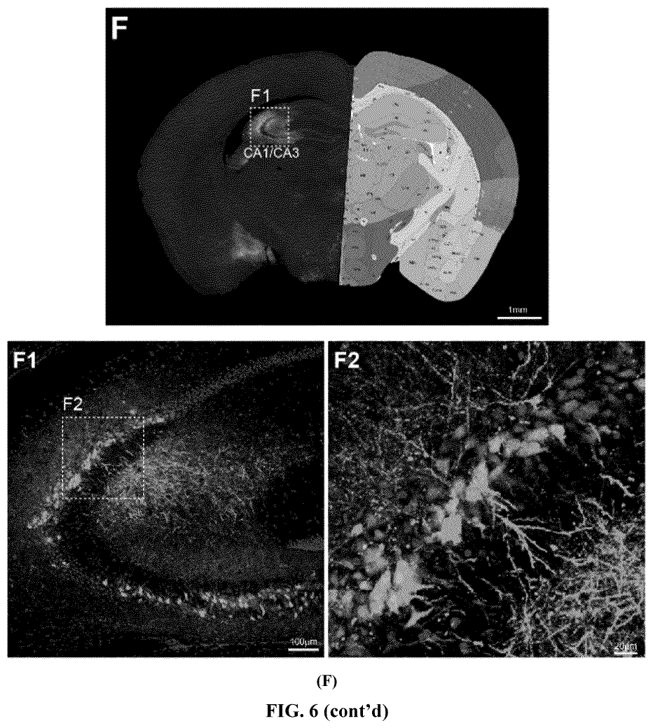

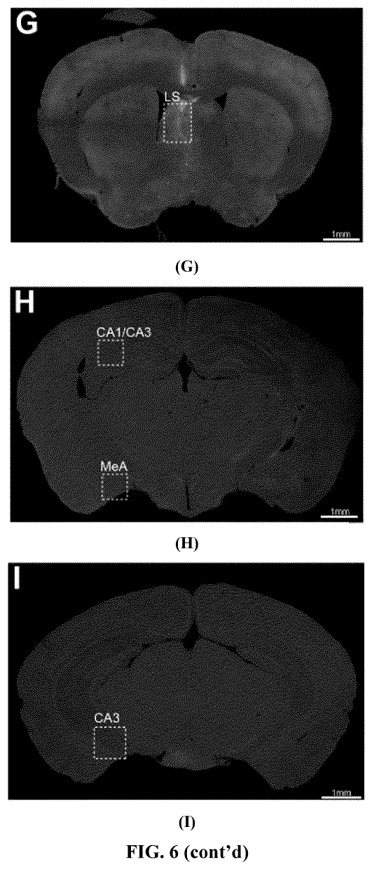

The lateral septal nucleus (LS) projections pathways were mapped (FIG. 6A). The LS (AP: +0.74 mm; ML: −0.30 mm; DV: −3.58 mm) was chosen as the injection site, where abundant GAD2 positive neurons are present. AAV2/9-DIO-mCh-gK (1.0×10¹² vg/ml, 150 nl) and H129-dgK-G4 (5.0×10⁸pfu/ml, 150 nl) were sequentially injected into the LS of GAD2-Cre mice on day 1 and day 22 respectively. On day 27, the brains were obtained after perfusion and processed for imaging (FIG. 6B). Neurons co-labeled with mCherry and GFP were observed around the injection site, representing the potential starter neurons that were infected by both viruses (FIG. 6C, indicated with white arrows). Abundant neurons in the downstream brain regions were observed, such as the MeA (FIG. 6D), and the CA3/CA1 of the hippocampus (FIG. 6E and 6F). Injecting H129-dgK-G4 alone only labeled neurons around the injection sites of LS (FIG. 6G), but not the connected regions (FIG. 6H and 6I), confirming the specificity of anterograde monosynaptic tracing.

These results demonstrated that the gKₘᵤₜ pseudotyped tracer H129-dgK-G4 is capable of performing specific anterograde monosynaptic tracing with strong labeling intensity and tracing efficiency, both in wildtype mice and in a starter specific manner in Cre-transgenic mice.

Brain diseases may be associated with neural network abnormalities. Alzheimer's disease and Parkinson's disease usually display neuronal damage and are accompanied by neural circuit changes, and autism is usually associated with neural network abnormalities. So dissecting the differences of neuronal connections between the diseased and healthy individuals is important for understanding the mechanisms of these diseases. The anterograde polysynaptic tracer H129-G4 was previously used to reveal the impaired connectivity from primary motor cortex (M1) to the subthalamic nucleus (STN) in unilateral 6-hydroxydopamine (6-OHDA)-lesioned parkinsonian rats. However, since H129-G4 is a polysynaptic tracer, the comparison had to be performed by limiting the transmission time to avoid potential detoured tracing, which is not capable of precisely controlling the transmission order. H129-dgK-G4 offers a better tool to achieve a more accurate comparison by its monosynaptic tracing specificity.

3×Tg-AD mouse is a broadly-used Alzheimer's disease model, which contains three mutations (APP Swedish, MAPT P301L, and PSEN1 M146V) associated with familial Alzheimer's disease. At the age of 3-month-old, the 3×Tg-AD mouse showed a significantly decrease level of synaptophysin in the cortex. To compare the synaptic connectivity between the cortex and other brain regions, we sequentially injected the AAV2/9-mCh-gK (1.0×10¹² vg/ml, 150 nl) and H129-dgK-G4(5.0×10⁸ pfu/ml. 150 nl) into the medial prefrontal cortex (mPFC, AP: +1.78 mm; ML: −0.16 mm; DV: −3.00 mm) of 3-month-old 3×Tg-AD or the control wildtype C57BL/6 mice at the same age (FIG. 7A). The labeled cells were quantitated in the cortical amygdaloid nucleus (CoA), which is one direct innervating region of mPFC. The representative images clearly showed fewer CoA neurons were labeled by H29-dgK-G4 in 3×Tg-AD than those in the wildtype mice (FIG. 7B). We counted the number of GFP-labeled CoA neurons in the position-matching brain slices in each group. In wildtype mice, an average of 272±38 GFP± neurons were observed in CoA of each mouse brain slice, whereas there are only 52±54 GFP⁺ neurons in CoA of each of the 3×Tg-AD mouse brain slice (FIG. 7C). Notably, the amount of the starter neurons in mPFC coinfected by H129-dgK-G4 and helper AAV were slightly less in 3×Tg-AD mice than in wildtype control (76±4 vs 86±2), the difference is not statistically significant. But, when normalizing the GFP⁺CoA neurons to the mPFC starter neuron in each mouse, 3×Tg-AD mouse clearly showed a significantly decreased mPFC-CoA connection (FIG. 7D). These results indicate that the mPFC-CoA connectivity in this Alzheimer's disease model at the age of 3-month old was decreased by 81% compared to the wildtype control, which is also consistent with the reduced cortex synaptophysin of the 3×Tg-AD mouse. Therefore, H129-dgK-G4 could be a potential powerful tool to quantify the direct neuronal connectivity.

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the scope of the present invention. Accordingly, the scope of the present invention is defined by the appended claims and is supported by the foregoing description.

### REFERENCES

David A T, Saied A, Charles A, Subramanian R, Chouljenko V N, Kousoulas K G: A herpes simplex virus 1 (McKrae) mutant lacking the glycoprotein K gene is unable to infect via neuronal axons and egress from neuronal cell bodies. mBio 2012, 3:e00144-00112.
Dong X, Zhou J, Qin H B, Xin B, Huang Z L, Li Y Y, Xu X M, Zhao F, Zhao C J, Liu J J, et al: Anterograde Viral Tracer Herpes Simplex Virus 1 Strain H129 Transports Primarily as Capsids in Cortical Neuron Axons. Journal of virology 2020, 94.
Yang B, Liu X J, Yao Y, Jiang X, Wang X Z, Yang H, Sun J Y, Miao Y, Wang W, Huang Z L, et al: WDR5 Facilitates Human Cytomegalovirus Replication by Promoting Capsid Nuclear Egress. Journal of virology 2018, 92.
Yang H, Xiong F, Song Y G, Jiang H F, Qin H B, Zhou J, Lu S, Grieco S F, Xu X, Zeng W B, et al: HSV-1 11129-Derived Anterograde Neural Circuit Tracers: Improvements, Production, and Applications. Neurosci Bull 2020.
Yang Y, Chen J, Guo Z, Deng S, Du X, Zhu S, Ye C, Shi Y S, Liu J J: Endophilin A1 Promotes Actin Polymerization in Dendritic Spines Required for Synaptic Potentiation. Front Mol Neurosci 2018, 11:177.
Zeng W B, Jiang H F, Gang Y D, Song Y G, Shen Z Z, Yang H, Dong X, Tian Y L, Ni R J, Liu Y, et al: Anterograde monosynaptic transneuronal tracers derived from herpes simplex virus 1 strain H129. Mol Neurodegener 2017, 12:38.

SEQUENCE LISTING

SEQ ID NO. 1: Gene sequence of wildtype H129 gK
(gK_{wt})
Atgctcgccgtccgttccctgcagcacctctcaaccgtcgtcttgataac
ggcgtacggcctcgtgctcgtgtggtacaccgtcttcggtgccagtccgc
tgcaccgatgtatttacgcggtacgccccaccggcaccaacaacgacacc
gccctcgtgtggatgaaaatgaaccagaccctattgtttctgggggcccc
gacgcacccccccaacgggggctggcgcaaccacgcccatatctgctacg
ccaatcttatcgcgggtagggtcgtgcccttccaggtcccacccgacgcc
atgaatcgtcggatcatgaacgtccacgaggcagttaactgtctggagac
cctatggtacacacgggtgcgtctggtggtcgtagggtggttcctgtatc
tggcgttcgtcgccctccaccaacgccgatgtatgtttggtgtcgtgagt
cccgcccacaagatggtggccccggccacctacctcttgaactacgcagg
ccgcatcgtatcgagcgtgttcctgcagtacccctacacgaaaattaccc
gcctgctctgcgagctgtcggtccagcgacagaacctggttcagttgttt
gagacggacccggtcaccttcttgtaccaccgccccgccatcggggtcat
cgtaggctgcgagttgatgctacgctttgtggccgtgggtctcatcgtcg
gcaccgctttcatatcccgtggggcatgtgcgatcacataccccctgttt
ctgaccatcaccacctggtgttttgtctccaccatcggcctgacagagct
gtattgtattctgcggcggggcccggcccccaagaacgcagacaaggccg
ccgccccggggcgatccaaggggctgtcgggcgtctgcgggcgctgttgt
tccatcatcctctcgggcatcgcagtgcgattgtgttatatcgccgtggt
ggccggggtggtgctcgtggogcttcactacgagcaggagatccagaggc
gcctgtttgatgtatga
SEQ ID NO. 2: Amino acid sequence of wildtype H129
gK (gK_{wt})
MLAVRSLQHLSTVVLITAYGLVLVWYTVFGASPLHRCIYAVRPTGTNNDT
ALVWMKMNQTLLFLGAPTHPPNGGWRNHAHICYANLIAGRVVPFQVPPDA
MNRRIMNVHEAVNCLETLWYTRVRLVVVGWFLYLAFVALHQRRCMFGVVS
PAHKMVAPATYLLNYAGRIVSSVFLQYPYTKITRLLCELSVQRQNLVQLF
ETDPVTFLYHRPAIGVIVGCELMLRFVAVGLIVGTAFISRGACAITYPLF
LTITTWCFVSTIGLTELYCILRRGPAPKNADKAAAPGRSKGLSGVCGRCC
SIILSGIAVRLCYIAVVAGVVLVALHYEQEIQRRLFDV*
SEQ ID NO. 3: Gene sequence of mutant H129 gK
(gKₘᵤₜ)
Atgctcgccgtccgttccctgcagcacctctcaaccgtcgtcttgataac
ggcgtacggcctcgtgctcgtgtggtacaccgtcttcggtgccagtccgc
tgcaccgatgtatttacgtggtacgccccaccggcaccaacaacgacacc
gccctcgtgtggatgaaaatgaaccagaccctattgtttctgggggcccc
gacgcacccccccaacgggggctggcgcaaccacgcccacatctcgtacg
ccaatctgatagcggggagggtcgtgcccttccaggtcccacccgacgcc
atgaatcgtcggatcatgaacgtccacgaggccgtcaactgtctggagac
cctatggtacacacgggtgcgtctggtggtcgtagggtggttcctgtatc
tggcgttcgtcgccctccaccaacgccgatgtatgtttggcgtcgtgagt
cccgcccacaagatggtggccccggccacctacctcttgaactacgcagg
ccgcatcgtatcgagcgtgttcctgcagtacccctacacgaaaattaccc
gcctgctctgcgagctgtcggtccagcggcaaaacctggttcagttgttt
gagacggacccggtcaccttcttgtaccaccgccccgccatcggggtcat
cgtgggctgcgagttgatcgtacgctttgtggccgtgggtctcatcgtcg
gcaccgctttcatatcccggggggcatgtgcgatcacataccccctgttt
ctgaccatcaccacctggtgttttgtctccaccatcggcctgacagagct
gtattgtattctgcggcggggcccggcccccaagaacgcagacaaggccg
ccgccccggggcgatccaaggggctgtcgggcgtctgcgggcgctgttgt
tccatcatcctgtcgggcatcgcaatgcgattgtgttatatcgccgtggt
ggccggggtggtgctcgtggcgcttcactacgagcaggagatccagaggc
gcctgtttgatgtatga
SEQ ID NO. 4: Amino acid sequence of mutant H129
gK (gKₘᵤₜ)
MLAVRSLQHLSTVVLITAYGLVLVWYTVFGASPLHRCIYVVRPTGTNNDT
ALVWMKMNQTLLFLGAPTHPPNGGWRNHAHISYANLIAGRVVPFQVPPDA
MNRRIMNVHEAVNCLETLWYTRVRLVVVGWFLYLAFVALHQRRCMFGVVS
PAHKMVAPATYLLNYAGRIVSSVFLQYPYTKITRLLCELSVQRQNLVQLF
ETDPVTFLYHRPAIGVIVGCELIVRFVAVGLIVGTAFISRGACAITYPLF
LTITTWCFVSTIGLTELYCILRRGPAPKNADKAAAPGRSKGLSGVCGRCC
SIILSGIAMRLCYIAVVAGVVLVALHYEQEIQRRLFDV*
SEQ ID NO. 5:
gK_{wt} primers: F - TCG AGG AGA ATC CTG GCC CAA TGC
TCG CCG TCC GTT CCC TG
SEQ ID NO. 6:
gK_{wt} primers: R - TCC GAT TTA AAT TCG AAT TCT CAT
ACA TCA AAC AGG CGC CTC TG

What is claimed is:

1. An anterograde monosynaptic transneuronal viral tracer system for mapping the direct postsynaptic targets of neurons in a given brain nucleus, comprising:
a tracer H129-derived recombinant HSV-1 virion; and
a helper AAV2/9-derived recombinant AAV2/9 virion with a recombinant AAV2/9 viral genome containing an HSV-1 wild-type gK encoding sequence;
wherein the tracer H129-derived recombinant HSV-1 virion comprises a recombinant HSV-1-H129 viral genome with an impaired gK gene, and a mutant gK protein that pseudotypes the tracer H129-derived recombinant HSV-1 virion.

2. The anterograde monosynaptic transneuronal viral tracer system of claim 1, wherein in the recombinant HSV-1-H129 viral genome with an impaired gK gene, the impaired gK gene is that gK-coding gene (UL53) is deleted.

3. The anterograde monosynaptic transneuronal viral tracer system of claim 1, wherein in the recombinant HSV-1-H129 viral genome with an impaired gK gene, the impaired gK gene is that gK-coding gene is replaced with resistance peptide-encoding sequence.

4. The anterograde monosynaptic transneuronal viral tracer system of claim 3, wherein the resistance peptide-encoding sequence includes Zeo^{R} and Amp^{R}.

5. The anterograde monosynaptic transneuronal viral tracer system of claim 1, wherein the tracer H129-derived recombinant HSV-1 virion is prepared by:
propagating the recombinant HSV-1-H129 viral genome with an impaired gK gene in Vero cells expressing wild-type H129 gK (gK_{wt}) protein to prepare a seed H129-derived recombinant HSV-1 virion with the gK_{wt} protein (H129-dgK(gK_{wt})); and
propagating the seed H129-dgK(gK_{wt}) in Vero cells expressing the mutant gK (gKₘᵤₜ) protein to prepare the tracer H129-derived recombinant HSV-1 virion (H129-dgK(gKₘᵤₜ))

6. The anterograde monosynaptic transneuronal viral tracer system of claim 5, wherein the mutant gK protein has an amino acid sequence represented by SEQ ID NO. 2 in which at least 5 point mutations including A40V, C82S, M223I, L224V, V309M are present.

7. The anterograde monosynaptic transneuronal viral tracer system of claim 6, wherein the gKₘᵤₜ protein has an amino acid sequence represented by SEQ ID NO. 4.

8. The anterograde monosynaptic transneuronal viral tracer system of claim 1, wherein the recombinant HSV-1-H129 viral genome with an impaired gK gene further comprises a first expression cassette that contains a first neuronal cell-specific promoter, a first fluorescent protein-encoding sequence.

9. The anterograde monosynaptic transneuronal viral tracer system of claim 8, wherein the first neuronal cell-specific promoter includes CMV promoter, SV40 promoter, CAG promoter, EF1a promoter, TH promoter, and Syn1 promoter.

10. The anterograde monosynaptic transneuronal viral tracer system of claim 8, wherein the first expression cassette further comprises a first linker and a second fluorescent protein-encoding sequence, where the first linker is disposed between the first and second fluorescent protein-encoding sequences.

11. The anterograde monosynaptic transneuronal viral tracer system of claim 10, wherein the first fluorescent protein-encoding sequence encodes a membrane-bound fluorescent protein.

12. The anterograde monosynaptic transneuronal viral tracer system of claim 10, wherein the recombinant HSV-1-H129 viral genome with an impaired gK gene further comprises a second expression cassette that contains a second neuronal cell-specific promoter, a third fluorescent protein-encoding sequence.

13. The anterograde monosynaptic transneuronal viral tracer system of claim 12, wherein the second expression cassette further comprises a second linker and a fourth fluorescent protein-encoding sequence, where the second linker is disposed between the third and fourth fluorescent protein-encoding sequences.

14. The anterograde monosynaptic transneuronal viral tracer system of claim 13, wherein the third fluorescent protein-encoding sequence encodes a membrane-bound fluorescent protein.

15. The anterograde monosynaptic transneuronal viral tracer system of claim 12, wherein the second neuronal cell-specific promoter includes CMV promoter, SV40 promoter, CAG promoter, EF1a promoter, TH promoter, and Syn1 promoter.

16. The anterograde monosynaptic transneuronal viral tracer system of claim 1, wherein the recombinant AAV2/9 viral genome containing an HSV-1 wild-type gK encoding sequence comprises a third expression cassette that contains a third neuronal cell-specific promoter, the wild-type gK-encoding sequence, a linker peptide-encoding sequence, and a fifth fluorescent protein-encoding sequence.

17 A method of preparing a tracer H129-derived recombinant HSV-1 virion, said method comprising:
propagating a recombinant HSV-1-H129 viral genome with an impaired gK gene in Vero cells expressing wild-type H129 gK (gK_{wt}) protein to prepare a seed H129-derived recombinant HSV-1 virion with the gK_{wt} protein (H129-dgK(gK_{wt})); and
propagating the seed H129-dgK(gK_{wt}) in Vero cells expressing the mutant gK (gKₘᵤₜ) protein to prepare the tracer H129-derived recombinant HSV-1 virion (H129-dgK(gKₘᵤₜ)

18. The method of claim 17, wherein the mutant gK protein has an amino acid sequence represented by SEQ ID NO. 2 in which at least 5 point mutations including A40V, C82S, M223I, L224V, V309M are present

19. The method of claim 18, wherein the gKₘᵤₜ protein has an amino acid sequence represented by SEQ ID NO. 4.
